(19) United States
(12) Patent Application Publication
(10) Pub. No.: US 2024/0002589 A1
(43) Pub. Date: Jan. 4, 2024

(54) POLYESTER FILM AND MANUFACTURING METHOD FOR THE SAME

(71) Applicant: SK microworks Co., Ltd., Suwon-si (KR)

(72) Inventors: Byeong Jae LIM, Suwon-si (KR); Young Min HEO, Suwon-si (KR); Chul Kyu KIM, Suwon-si (KR)

(73) Assignee: SK microworks Co., Ltd., Suwon-si (KR)

(21) Appl. No.: 18/216,840

(22) Filed: Jun. 30, 2023

(30) Foreign Application Priority Data
Jun. 30, 2022 (KR) 10-2022-0080633

(51) Int. Cl. C08G 63/199; C08J 5/18

(52) U.S. Cl. CPC C08G 63/199; C08J 5/18; C08J 2367/02; B29C 48/08

(57) ABSTRACT

A polyester film according to an embodiment includes a polyester resin. The polyester resin includes a repeating unit having a cyclohexane skeleton and a repeating unit having a benzene ring. The polyester film has a base peak related to bending vibration of the benzene ring and a first peak related to C—O stretching vibration in a spectrum measured using a Fourier transform infrared spectrometer (FT-IR). The base peak is a peak at a wavenumber of 788 cm⁻¹ to 798 cm⁻¹. The first peak is a peak at a wavenumber of 1129 cm⁻¹ to 1139 cm⁻¹. The intensity of the base peak and the intensity of the first peak differ by −0.05 to +0.05. According to an embodiment, a polyester resin having a controlled degree of crystallization can be applied to a film to impart excellent heat resistance, mechanical properties, and the like to the film.

### CROSS-REFERENCE TO RELATED APPLICATION

This application claims the benefit under 35 USC § 119(a) of Korean Patent Application No. 10-2022-0080633, filed on Jun. 30, 2022, the entire disclosure of which is incorporated herein by reference for all purposes.

### BACKGROUND

### 1. Field

Embodiments relate to a polyester film and the like.

### 2. Discussion of Related Art

Polyesters are stable in terms of heat resistance, mechanical strength, transparency, and the like. Therefore, polyesters have been widely used in various fields such as containers, packaging materials, films, cables, and the like. Particularly, polycyclohexylenedimethylene terephthalate (PCT) has excellent heat resistance, moisture resistance, and the like compared to other resins, and the application thereof is gradually increasing in fields that require high heat resistance, such as electric vehicles and electric parts.

PCT resins are polyester resins with a cyclohexane skeleton, which can be produced by condensation polymerization of cyclohexanedimethanol and terephthalic acid.

PCT resins have high crystallization characteristics. These characteristics can help improve processability and production efficiency in the manufacture of typical thick injection-molded articles. However, in the case in which an extrusion process is applied in a manufacturing process as a sheet or film manufacturing process, when the crystallization characteristics of polymers are not controlled well, product quality or production yield is easily degraded.

The above-described background art is technical information that the inventor possesses for deriving the present invention or acquired in the derivation process, and cannot necessarily be said to be known technology disclosed to the general public prior to the filing of the present invention.

### SUMMARY

In one general aspect, a polyester film according to one embodiment is a film including a polyester resin.

The polyester resin includes a repeating unit having a cyclohexane skeleton and a repeating unit having a benzene ring.

The polyester film has a base peak which is a peak related to bending vibration of the benzene ring and a first peak which is a peak related to C—O stretching vibration in a spectrum measured using a Fourier transform infrared spectrometer (FT-IR).

The base peak is a peak at a wavenumber of 788 cm⁻¹ to 798 cm⁻¹.

The first peak is a peak at a wavenumber of 1129 cm⁻¹ to 1139 cm⁻¹.

An intensity of the base peak and an intensity of the first peak differ by −0.05 to +0.05.

The polyester film may have a second peak which is a peak distinguished from the first peak and related to C—O stretching vibration in the spectrum.

The second peak may be a peak at a wavenumber of 810 cm⁻¹ to 820 cm⁻¹.

The intensity of the base peak and the intensity of the second peak may differ by −0.05 to +0.05.

The polyester film may have a third peak which is a peak related to C═O—O stretching vibration in the spectrum.

The third peak may be a peak at a wavenumber of 850 cm⁻¹ to 860 cm⁻¹.

The intensity of the base peak and an intensity of the third peak may differ by −0.05 to +0.05.

The polyester film may have a fourth peak which is a peak related to C═O—O stretching vibration in the spectrum.

The fourth peak may be a peak at a wavenumber of 966 cm⁻¹ to 976 cm⁻¹.

The intensity of the base peak and the intensity of the fourth peak may differ by −0.05 to +0.05.

The polyester film may have a tensile strength of 7 kgf/mm² or more as measured after being left at 250° C. for 30 minutes.

The polyester film may have an elongation rate of 100% or more as measured after being left at 250° C. for 30 minutes.

The polyester film may have a haze value of 15% or less as measured after being left at 250° C. for 30 minutes.

The polyester film may have a yellow index of 3 or less as measured after being left at 250° C. for 30 minutes.

The repeating unit having a benzene ring may include a dicarboxylic acid-based repeating unit.

The dicarboxylic acid-based repeating unit may include a terephthalic acid-based repeating unit and an isophthalic acid-based repeating unit.

The polyester resin may have a weight-average molecular weight of 30,000 g/mol to 50,000 g/mol.

A method of manufacturing a polyester film according to another embodiment includes: extruding a polyester resin composition to prepare a sheet (extrusion step); stretching the sheet to prepare a film before thermal treatment (stretching step); and thermosetting the film before thermal treatment to prepare a polyester film (thermosetting step).

The polyester film includes a polyester resin.

The polyester resin includes a repeating unit having a cyclohexane skeleton and a repeating unit having a benzene ring.

The polyester film has a base peak which is a peak related to bending vibration of the benzene ring and a first peak which is a peak related to C—O stretching vibration in a spectrum measured using a Fourier transform infrared spectrometer (FT-IR).

The base peak is a peak at a wavenumber of 788 cm⁻¹ to 798 cm⁻¹.

The first peak is a peak at a wavenumber of 1129 cm⁻¹ to 1139 cm⁻¹.

The polyester film has a difference in intensities of the base peak and the first peak of −0.05 to +0.05.

In at least one of the extrusion step, stretching step, and thermosetting step, a process condition for adjusting the crystallization characteristic of the polyester film is set based on the spectrum of the polyester film measured using a Fourier transform infrared spectrometer.

The stretching step may include a longitudinal stretching process of longitudinally stretching the sheet to prepare a longitudinally stretched sheet and a lateral stretching process of laterally stretching the longitudinally stretched sheet to prepare a film before thermal treatment.

In the stretching step, stretching ratios of the longitudinal stretching process and the lateral stretching process for adjusting the crystallization characteristic of the polyester film may be set based on the spectrum of the polyester film measured using a Fourier transform infrared spectrometer.

The stretching ratio of the longitudinal stretching process may be in a range of 2 to 4, and the stretching ratio of the lateral stretching process may be in a range of 3 to 5.

In the thermosetting step, a thermosetting temperature for adjusting the crystallization characteristic of the polyester film may be set based on the spectrum of the polyester film measured using a Fourier transform infrared spectrometer.

The thermosetting temperature may be 200° C. to 250° C.

### DETAILED DESCRIPTION

Hereinafter, the present invention will be described in detail with reference to embodiments so that those skilled in the art can easily carry out the present invention. However, the present invention may be embodied in several different forms, and therefore, is not limited to the embodiments described herein.

As used herein, the term “about” or “substantially” is intended to have meanings close to numerical values specified with an allowable error and intended to prevent accurate or absolute numerical values disclosed for understanding of the present invention from being illegally or unfairly used by any unscrupulous third party.

Throughout this specification, the term “combination thereof” included in the expression of the Markush form means a mixture or combination of one or more selected from the group consisting of the components described in the expression of the Markush form, and it means including one or more selected from the group consisting of the above components.

Throughout this specification, description of “A and/or B” means “A, B, or A and B.”

Throughout this specification, terms such as “first” and “second” or “A” and “B” are used to distinguish the same terms from each other unless otherwise specified.

In this specification, the expression “B is located on A” means that B is located on A, or B is located on A while another layer is located therebetween, and it is not interpreted as being limited to B being located so as to come into contact with the surface of A.

In this specification, a singular expression is interpreted as a meaning including a singular number or a plurality interpreted in the context unless otherwise specified.

In this specification, room temperature refers to any one temperature of 20° C. to 25° C.

In this specification, a resin is interpreted as a meaning including a resin itself and a compound derived from the resin. For example, a polyester resin described in this specification is interpreted as a meaning including a polyester resin and a derivative of the polyester resin.

In this specification, a “-based repeating unit” refers to a repeating unit derived from a monomer compound. For example, a diol-based repeating unit refers to a repeating unit derived from a diol-based compound.

A confirmation method presented in this specification utilizes the results of an analysis device, and there may be a slight difference in the characteristic wavenumber and its intensity described below due to a difference in the device itself, the type thereof, or the program loaded on the device. However, the invention presented in this specification should not be interpreted based on the above fact.

Hereinafter, an embodiment will be described in further detail.

A film formation process of a polymer resin is performed in a series of steps such as melting (or softening), sheet formation, temperature elevation, stretching, thermosetting, and the like. The degree of crystallization of a polymer resin affects manufacturing yield and also affects the properties of the manufactured film, such as mechanical properties, optical properties, and the like.

The adjustment of the degree of crystallization of a polymer resin in each step is important in terms of an improvement in yield and quality control of the manufactured article. To this end, it is necessary to confirm or predict the degree of crystallization of a polymer resin in each step. Particularly, this is more necessary in the case of a polymer resin in which crystallization proceeds rapidly.

As a result of applying various methods as a method for more easily confirming or predicting the degree of crystallization of a polyester film including a repeating unit having a cyclohexane skeleton, the inventors present a method utilizing a Fourier transform infrared spectrometer of an embodiment that is judged to reflect the specificity of a resin well.

Method I of Checking Degree of Crystallization of Polymer

In order to accomplish the above objective, a method of checking the degree of crystallization of a polymer according to an embodiment includes: a preparation step; a measurement step; a selection step; a search step; and an output step.

The preparation step is a step for preparing a sample containing a polymer to be checked.

The sample includes a polyester resin, and the polyester resin includes a repeating unit having a cyclohexane skeleton.

The polyester resin may include polycyclohexylenedimethylene terephthalate.

Since the polyester resin has a very high crystallization rate compared to a polyester resin having an ethylene glycol residue, it is not easy to control the degree of crystallization.

The sample may include a stretched film.

The sample may include a uniaxially stretched film.

The sample may include a biaxially stretched film.

The sample may include a sequentially biaxially stretched film.

In the polymer resin included in the stretched film, crystallization promoted by heat may be occurred. In addition, in the polymer resin included in the stretched film, crystallization promoted by stretching (machine direction (MD) stretching and transverse direction (TD) stretching in the case of a biaxially stretched film) may be generated.

The preparation step is a step for preparing a sample containing a polymer to be identified, and a sample adjusted to have a size measurable by a spectrometer may be prepared as necessary.

It is convenient to prepare a sample cut into a typical quadrangular shape, but the present invention is not limited thereto.

The sample may be prepared to have a surface parallel or perpendicular to a stretching direction. For example, the sample may be prepared by cutting so that the machine direction of a stretched film and one surface of the sample are parallel. Also, the sample may be prepared by cutting so that the transverse direction of a stretched film and one surface of the sample are parallel. The sample prepared so that one surface of the sample is parallel to the machine direction or transverse direction of a stretched film is favorable for checking the degree of crystallization promoted by stretching to be described below.

The sample may be prepared into a quadrangular shape having a length and width of 5 to 6 cm, but the present invention is not limited thereto.

The measurement step is a step of obtaining spectrum information including information on absorbance intensity at a wavenumber at which crystallization characteristics of the sample are exhibited using a spectrometer.

As the spectrometer, a Fourier transform infrared spectrometer (FT-IR) may be used.

As the FT-IR instrument, a UMA 600 product manufactured by Varian may be used, but the present invention is not limited thereto. A specific method of setting the FT-IR instrument follows the description of the experimental example to be described below. For example, measurement may be made by applying diamond and Ge single bounce ATR and conditions of an MCT detector, resolution of 4, and #32 scans.

A wavenumber range in measurement may include all wavenumbers provided by the instrument and may be from about 800 to about 1200 cm⁻¹, and one or more of a first wavenumber, a second wavenumber, a third wavenumber, and a fourth wavenumber to be described may be optionally selected.

When information on a degree of crystallization, which is to be obtained from the spectrum information, is for the degree of crystallization of the polymer resin by heat, the spectrum information may be obtained by averaging the results of two measurements while rotating the sample about 90°. Specifically, the sample has a first measurement value measured for a sample placed in a first direction and a second measurement value measured for the sample placed in a second direction, the first direction and the second direction are substantially perpendicular to each other, and the spectrum information or the characteristic corresponds to an average value of the first measurement value and the second measurement value.

When information on a degree of crystallization, which is to be obtained from the spectrum information, is for the degree of crystallization of the polymer resin by MD stretching and/or TD stretching, the spectrum information is obtained using a polarizing plate. Specifically, the light beam of the spectrometer passes through a polarizing plate and is incident on the sample, the direction of wave of the light beam is parallel to the surface of the sample, and the traveling direction of the light beam is perpendicular to the sample. A polarizing plate set to 90° is used. As the sample, a sample cut so that at least one side of the sample is parallel to the machine direction of a stretched film may be used. Alternatively, as the sample, a sample cut so that at least one side of the sample is parallel to the transverse direction of a stretched film may be used. Information on a degree of crystallization checked by measurement performed by varying the direction of the sample shows a degree of MD orientation crystallization and a degree of TD orientation crystallization, and they may show mutually different results.

The selection step is a step of selecting a feature to be used as a search key from the spectrum information. The spectrum information may usually be a table or graph (graph in which the X axis is the wavenumber and the Y axis is the absorption spectrum intensity) in which absorption spectrum intensity versus wavenumber is presented. In an embodiment, as the spectrum information used as a search key corresponding to the degree of crystallization of a polymer-containing sample, at least one of four intensities (first to fourth intensities) to be described below is presented.

For example, when it is intended to check the degree of crystallization of a sample by heat, a search key is the spectrum intensity at a first wavenumber, for example, the spectrum intensity of a peak positioned at a first wavenumber. The first wavenumber may be in a range of about 1129 cm⁻¹ to about 1139 cm⁻¹ or about 1132 cm⁻¹ to about 1136 cm⁻¹. Specifically, the first wavenumber may be around 1134 cm⁻¹. The peak intensity at the first wavenumber is referred to as a first intensity.

The peak is considered to be a peak related to C—O stretching and results from modification of a repeating unit having a cyclohexane skeleton due to heat-induced crystallization. The position of the first peak is expressed as being near the corresponding wavenumber considering that a peak shift may occur depending on measurement conditions, data processing conditions, and sample conditions, and the like. Even when the expression of the “near” is not described, it is interpreted as including the “near” (the same as below).

The predicted value of the degree of crystallization of a sample by heat is a result obtained by searching for information on the degree of crystallization corresponding to a spectrum intensity value in the spectrum database, which has an equivalence condition of a predetermined value or more relative to an input value, when the spectrum intensity at the first wavenumber is used as the input value.

In addition, the predicted value of the degree of crystallization of a sample by heat may be confirmed with an intensity value relative to the peak intensity at a base wavenumber in the same sample.

For example, the base wavenumber may be about 793 cm⁻¹ or in a range of about 788 cm⁻¹ to 798 cm⁻¹. The base wavenumber is related to the bending vibration of a benzene ring appearing at 780 cm⁻¹ to 800 cm⁻¹, and since the base wavenumber does not change significantly depending on heat or orientation, it is determined that the base wavenumber can be applied as a reference in evaluation of the relative intensity of a spectrum (the same as below).

In the predicted value of the degree of crystallization by heat for a sample, a difference between a first intensity value and a peak at the base wavenumber (referred to as a base peak) may be within a certain range. For example, the first intensity value and the base intensity may differ by −0.05 to +0.05, and specifically, −0.02 to +0.02. In this case, it can be determined that the range is within a range of thermal crystallization suitable for processing the resin in the form of a film.

For example, when it is intended to check the degree of crystallization by heat for a sample, a search key is the spectrum intensity at a second wavenumber, for example, the spectrum intensity of a peak positioned at a second wavenumber. The second wavenumber may be in a range of 810 cm⁻¹ to 820 cm⁻¹ or 812 cm⁻¹ to 817 cm⁻¹. Specifically, the second wavenumber may be around 815 cm⁻¹. The peak intensity at the second wavenumber is referred to as a second intensity.

The peak is considered to be a peak related to C—O stretching vibration and results from modification of repeating unit having a cyclohexane skeleton due to heat-induced crystallization.

The predicted value of the degree of crystallization by heat for a sample is a result obtained by searching for information on the degree of crystallization corresponding to a spectrum intensity value in the spectrum database, which has an equivalence condition of a predetermined value or more relative to an input value, when the spectrum intensity at the second wavenumber is used as the input value.

In addition, the predicted value of the degree of crystallization by heat for a sample may be confirmed with an intensity value relative to the intensity at a base wavenumber in the same sample.

In the base wavenumber or the base intensity, for example, the above-described peak or intensity may be applied.

The predicted value of the degree of crystallization by heat for a sample may have a second intensity value relative to the base intensity within a certain range. For example, the second intensity and the base intensity may differ by −0.05 to +0.05, and specifically, −0.02 to +0.02. In this case, it can be determined that the range is within a range of thermal crystallization suitable for processing the resin in the form of a film.

Crystallization of a sample according to orientation may be associated with a stretching process. Physical properties of a film may vary depending on how polymer chains are arranged to be parallel to each other in a polymer film, and this is associated with the degree of crystallization according to orientation. Also, the degree of crystallization according to orientation may be affected and controlled by a stretching process, but it is not interpreted as being limited to the degree of crystallization controlled only by a stretching process.

For example, when it is intended to confirm the degree of crystallization of a sample according to orientation, a search key is the spectrum intensity at a third wavenumber, for example, the spectrum intensity of a peak positioned at a third wavenumber. The third wavenumber may be in a range of 850 cm⁻¹ to 860 cm⁻¹ or 853 cm⁻¹ to 857 cm⁻¹. Specifically, the third wavenumber may be around 855 cm⁻¹. The peak intensity at the third wavenumber is referred to as a third intensity.

For example, when it is intended to confirm the degree of crystallization of a sample according to orientation, a search key is the spectrum intensity at a fourth wavenumber, for example, the spectrum intensity of a peak positioned at a fourth wavenumber. The fourth wavenumber may be in a range of 966 cm⁻¹ to 976 cm⁻¹ or 969 cm⁻¹ to 973 cm⁻¹. Specifically, the fourth wavenumber may be around 971 cm⁻¹. The peak intensity at the fourth wavenumber is referred to as a fourth intensity.

The third wavenumber and the fourth wavenumber are considered to be wavenumbers related to C═O—O stretching vibration. It is believed that a vibration change according to the orientation of the polymer resin is found at the wavenumbers.

For example, the predicted value of the degree of crystallization of a sample according to orientation is a result of searching for information on the degree of crystallization corresponding to a spectrum intensity value in the spectrum database, which has an equivalence condition of a predetermined value or more relative to the input value, when the spectrum intensity at the third wavenumber and/or fourth wavenumber is used as the input value.

In addition, the predicted value of the degree of crystallization of a sample according to orientation may be obtained from an intensity value relative to the base peak intensity in the same sample.

Since the description of the base peak is the same as that described above, a detailed description thereof is omitted.

In the predicted value of the degree of crystallization of a sample according to orientation, an intensity value(s) at the third wavenumber and/or fourth wavenumber relative to that of the base peak may be within a certain range. For example, the intensity value(s) at the third wavenumber and/or the fourth wavenumber and the base peak may differ by −0.05 to +0.05, and specifically, −0.02 to +0.02. In this case, it can be determined that the range is within a range of thermal crystallization suitable for processing the resin in the form of a film.

The spectrum database utilized in check of the degree of crystallization according to orientation is based on a result measured after the light incident on a sample in measurement passes through a polarizing plate. For convenience of measurement, a sample cut so that at least one side of the sample is parallel to the machine direction or transverse direction of the stretched film included in the sample may be applied.

It is possible to distinguish whether the predicted value is a degree of MD orientation crystallization or a degree of TD orientation crystallization depending on whether a direction of the light passing through a polarizing plate is parallel to the machine direction or transverse direction of the stretched film.

Specifically, the light beam of the spectrometer passes through a polarizing plate and is incident on a sample. The polarizing plate is mounted between an IR source and the sample and set to about 90°, and then the position thereof is adjusted so that an interferogram signal is maximized. Since the setup of equipment is the same as in the measurement of surface orientation in many aspects, the direction of wave of the light beam is generally parallel to the surface of the sample when the polarizing plate is set to 90°, and the light beam passing through the sample enables check of the MD crystallinity of the film perpendicular to the traveling direction of the film. Also, when the direction of wave of the light beam is parallel to the thickness of the sample by adjusting the polarizing plate, the light beam passing through the sample enables check of the TD crystallinity of the film perpendicular to the traveling direction of the film.

The search step is a step of searching for information on a degree of crystallization, which satisfies an equivalence condition of a predetermined value or more relative to the search key in the spectrum database.

In the spectrum database, the degree of crystallization of a polymer resin and spectrum characteristics corresponding to the degree of crystallization are recorded.

As the spectrum database utilized in check of the degree of crystallization by heat, a database obtained by averaging the results of two measurements while rotating the sample about 900 may be used.

The spectrum database utilized in check of the degree of crystallization by orientation may be a spectrum database obtained from a sample cut to have one surface of the sample to be parallel to a machine direction (or transverse direction) and by separately recording a MD measurement value and a TD measurement value using a polarizing plate.

The spectrum database has data in which the specific absorption spectrum intensity at the corresponding wavenumber indicates a certain level of crystallization, and based on the data, search for information on a degree of crystallization, which satisfies an equivalence condition of a predetermined value or more, is possible.

In this case, the spectrum database utilized in check of the degree of crystallization by orientation may be built separately from the spectrum database utilized in check of the degree of crystallization by heat. Also, as the search key for checking the degree of crystallization by orientation, both the absorbance spectrum intensity at a characteristic wavenumber, which is to be used as a search key, and whether it is in a machine direction or transverse direction may be selected.

The equivalence condition of a predetermined value or more is, for example, that an error range between the absorbance spectrum intensity at the corresponding wavenumber, which is a search key, and the absorbance spectrum intensity at the corresponding wavenumber in the spectrum database may be within 10%, 5%, or 1%, but the present invention is not limited thereto.

The equivalence condition of a predetermined value or more may be derived, for example, from a trend line (trend line of the degree of crystallization according to spectrum intensity) recorded in the spectrum database. In this case, in the equivalence condition, an R² of the trend line may be 0.9 or more, 0.95 or more, or 0.98 or more.

The output step is a step of outputting the information on a degree of crystallization. In this case, information on a degree of crystallization, which satisfies the equivalence condition in the spectrum database, is output, and the output may be a typically applied output on a screen or on an output object, but the present invention is not limited thereto.

The output may obtain whether information on a degree of crystallization is by heat or stretching in addition to information on crystallization. Also, in the case of information on the degree of crystallization by stretching, whether it is in a machine direction or transverse direction may also be output.

The method of checking the degree of crystallization of a polymer according to an embodiment can check or predict the degree of thermal crystallization or orientational crystallization of a sample by utilizing the absorbance spectrum intensity at a specific wavenumber in the spectrum information using a Fourier transform infrared spectrometer. This can provide basic information for controlling the degree of crystallinity in the process of manufacturing a polymer product in the form of a sheet or film, and a product having further controlled crystallinity can be more efficiently manufactured using the information.

Methods II and Ill of Checking Degree of Crystallization of Polymer

A method of checking the degree of crystallization of a polymer of a film according to another embodiment includes: a preparation step; a measurement step; a selection step; and a check step.

The preparation step is a step of preparing a film-type sample containing a polyester resin.

The polyester resin may include a repeating unit having a cyclohexane skeleton and a repeating unit having a benzene ring.

The polyester resin may include polycyclohexylenedimethylene terephthalate. The polyester resin may be polycyclohexylenedimethylene terephthalate.

The measurement step is a step of obtaining spectrum information by measuring the sample using a spectrometer.

As the spectrometer, a Fourier transform infrared spectrometer (FT-IR) may be used.

Since the descriptions of the sample preparation, spectrometer setting, and the like are the same as those described above, a detailed description thereof is omitted.

The selection step is a step of selecting a search wavenumber for confirming a feature applied at a base wavenumber and crystallization information in the spectrum information.

Generally, in spectrum information, wavenumber information is presented on the X axis, and spectrum intensity is presented on the Y axis. A peak is observed at a specific wavenumber, and it is possible to check the characteristics of a polymer corresponding to the peak.

An embodiment checks or predicts the degree of crystallization of the polymer resin in the film by associating these characteristics with crystallization characteristics of the film.

The base wavenumber may be related to bending vibration of a benzene ring.

The base wavenumber may be in a range of about 788 cm⁻¹ to 798 cm⁻¹.

Since the description of the base wavenumber is the same as that described above, a detailed description thereof is omitted.

The search wavenumber may be related to C—O stretching vibration or C═O—O stretching vibration.

The search wavenumber may be a first wavenumber in a range of 1129 cm⁻¹ to 1139 cm⁻¹, a second wavenumber in a range of 810 cm⁻¹ to 820 cm⁻¹, a third wavenumber in a range of 850 cm⁻¹ to 860 cm⁻¹, or a fourth wavenumber in a range of 966 cm⁻¹ to 976 cm⁻¹.

The peak related to C—O stretching vibration may appear at a first wavenumber of 1129 cm⁻¹ to 1139 cm⁻¹ and/or a second wavenumber of 810 cm-1 to 820 cm⁻¹.

The peak related to C═O—O stretching vibration may appear at a third wavenumber of 850 cm⁻¹ to 860 cm⁻¹ and/or a fourth wavenumber of 966 cm⁻¹ to 976 cm⁻¹.

Since the description such as referring to the peak intensities at the base wavenumber and the search wavenumber as base intensity and search intensity, respectively, is the same as that described above, a detailed description thereof is omitted.

The check step is a step of checking whether the peak intensity at the base wavenumber and the peak intensity at the search wavenumber differ by −0.05 to +0.05 or −0.02 to +0.02.

When the difference falls within the above-described range, the degree of crystallization of a polymer resin to be processed in the form of a film is appropriately controlled, and a film having improved mechanical properties and the like can be provided.

In an embodiment, information that helps control the degree of crystallization of a polymer resin to be processed in the form of a film may be provided.

Polyester Film

A polyester film according to still another embodiment includes a polyester resin.

The polyester resin includes a repeating unit having a cyclohexane skeleton and a repeating unit having a benzene ring.

The polyester film has a base peak which is a peak related to bending vibration of the benzene ring and a first peak which is a peak related to C—O stretching vibration in a spectrum measured using a Fourier transform infrared spectrometer (FT-IR).

The base peak is a peak at a wavenumber of 788 cm⁻¹ to 798 cm⁻¹. The base peak may be a peak at a wavenumber of 793 cm⁻¹.

The first peak is a peak at a wavenumber of 1129 cm⁻¹ to 1139 cm⁻¹. The first peak may be a peak at a wavenumber of 1132 cm⁻¹ to about 1136 cm⁻¹. The first peak may be a peak at a wavenumber of 1134 cm⁻¹.

The intensity of the base peak and the intensity of the first peak differ by −0.05 to +0.05. The intensities may differ by −0.02 to +0.02.

When the polyester film has such characteristics, the resin applied to the film may have thermal crystallization characteristics suitable for being processed in the form of a film. The resin may contribute to imparting excellent heat resistance, mechanical properties, and the like to the polyester film.

The polyester film may have a second peak which is a peak distinguished from the first peak and related to C—O stretching vibration in the spectrum.

The second peak may be a peak at a wavenumber of 810 cm⁻¹ to 820 cm⁻¹. The second peak may be a peak at a wavenumber of 812 cm⁻¹ to 817 cm⁻¹. The second peak may be a peak at 815 cm⁻¹.

The intensity of the base peak and the intensity of the second peak may differ by −0.05 to +0.05. The intensities may differ by −0.02 to +0.02.

In this case, excessive crystallization of the polyester resin by thermal treatment is suppressed, and thus the polyester film can secure both excellent heat resistance and excellent mechanical properties.

The polyester film may have a third peak which is a peak related to C═O—O stretching vibration in the spectrum.

The third peak may be a peak at a wavenumber of 850 cm⁻¹ to 860 cm⁻¹. The third peak may be a peak at 853 cm⁻¹ to 857 cm⁻¹. The third peak may be a peak at 855 cm⁻¹.

The intensity of the base peak and the intensity of the third peak may differ by −0.05 to +0.05. The intensities may differ by −0.02 to +0.02.

The polyester film having such characteristics can have excellent mechanical properties as the degree of orientation of the resin is appropriately adjusted.

The polyester film may have a fourth peak which is a peak related to C═O—O stretching vibration in the spectrum.

The fourth peak may be a peak at a wavenumber of 966 cm⁻¹ to 976 cm⁻¹. The fourth peak may be a peak at 969 cm⁻¹ to 973 cm⁻¹. The fourth peak may be a peak at 971 cm⁻¹.

The intensity of the base peak and the intensity of the fourth peak may differ by −0.05 to +0.05. The intensities may differ by −0.02 to +0.02.

The polyester film having such characteristics can have improved mechanical properties such as modulus and the like.

Since the descriptions of the method of measuring the spectrum of the polyester film using a Fourier transform infrared spectrometer (FT-IR), the base peak, the first peak, the second peak, the third peak, and the fourth peak are the same as those described above, a detailed description thereof is omitted.

The polyester resin includes a dicarboxylic acid-based repeating unit and a diol-based repeating unit.

The dicarboxylic acid-based repeating unit may include a terephthalic acid-based repeating unit and an isophthalic acid-based repeating unit.

The dicarboxylic acid-based repeating unit may include a terephthalic acid residue and an isophthalic acid residue.

The diol-based repeating unit includes a repeating unit having a cyclohexane skeleton.

The repeating unit having a cyclohexane skeleton may be a cyclohexanedimethanol residue.

The dicarboxylic acid-based repeating unit may include, based on a total of 100 mol %, 80 mol % or more or 90 mol % or more and 100 mol % or less of the terephthalic acid residue and 20 mol % or less, 15 mol % or less, 12 mol % or less, 10 mol % or less, 8 mol % or less, 6 mol % or less, or 4 mol % or less of the isophthalic acid residue. The polyester resin may include more than 0 mol %, 1 mol % or more, or 2 mol % or more of the isophthalic acid residue.

When the terephthalic acid residue and the isophthalic acid residue are included in the above-described amounts in the dicarboxylic acid-based repeating unit, relatively high melting point and low crystallization characteristics can be exhibited. Also, the inclusion of the above-described amount of the isophthalic acid residue can contribute to an improvement in the long-term durability of the polyester film.

The diol-based repeating unit may further include the following diol-based repeating units in addition to cyclohexanedimethanol. For example, ethylene glycol, 1,3-propanediol, 1,2-octanediol, 1,3-octanediol, 2,3-butanediol, 1,3-butanediol, 1,4-butanediol, 1,5-pentanediol, 2,2-dimethyl-1,3-propanediol (neopentyl glycol), 2-butyl-2-ethyl-1,3-propanediol, 2,2-diethyl-1,5-pentanediol, 2,4-diethyl-1,5-pentanediol, 3-methyl-1,5-pentanediol, 1,1-dimethyl-1,5-pentanediol, or a residue derived therefrom may be included.

The diol-based repeating unit may include, based on a total of 100 mol %, 70 mol % or more, 80 mol % or more, or 90 mol % or more and 100 mol % or less of the cyclohexanedimethanol residue.

The polyester resin may be a polycyclohexylenedimethylene terephthalate resin.

The polycyclohexylenedimethylene terephthalate resin may have a weight-average molecular weight (Mw) of 30,000 g/mol to 50,000 g/mol or 30,000 g/mol to 40,000 g/mol.

The polycyclohexylenedimethylene terephthalate resin may have a melting point of 280° C. or more. Since the melting point is higher than that of a polyethylene terephthalate resin, which is a polyester resin generally applied to a polyester film, excellent heat resistance can be imparted to the polyester film.

The polyester film may further include inorganic particles as necessary. The inorganic particles may be included in the polyester film while being mixed with the polyester resin.

As the inorganic particles, for example, any one selected from the group consisting of silica, talc, titanium oxide (TiO₂), calcium carbonate (CaCO₃), barium sulfate (BaSO₄), and a combination thereof may be included.

The application of the inorganic particles may help increase the mechanical strength of the polyester film.

The polyester film may include an antioxidant.

The antioxidant may be applied to prevent a resin exposed to high temperature from aging in a polyester resin synthesis process, but in an embodiment, the antioxidant is applied in a film formation process of a resin rather than in a resin synthesis process. Also, as the antioxidant, a mixture of at least three types of antioxidants may be used. Specifically, as the antioxidant, a phenol-based antioxidant, a phosphorus-based antioxidant, or a sulfur-based antioxidant may be included.

The phenol-based antioxidant may be, for example, tetrakis[methylene-3-(3,5-di-tert-butyl-4-hydroxyphenyl)propionate]methane; octadecyl-3-(3,5-di-tert-butyl-4-hydroxyphenyl)propionate; benzenepropanoic acid; 3,5-bis(1,1-dimethylethyl)-4-hydroxy alkyl ester (C7 or C9 alkyl); triethylene glycol-bis-3-(3-tert-butyl-4-hydroxy-5-methylphenyl)propionate; tris(3,5-di-tert-butyl-4-hydroxybenzyl)isocyanurate; 1,3,5-trimethyl-2,4,6-tris(3,5-di-tert-butyl-4-hydroxybenzyl)benzene; or a combination thereof.

The phosphorus-based antioxidant may be, for example, 3,9-bis(2,4-dicumylphenoxy)-2,4,8,10-tetraoxa-3,9-diphosphaspiro[5.5]undecane; bis(2,6-di-tert-butyl-4-methylphenyl)pentaerythritol-di-phosphite; bis(2,4-di-tert-butylphenyl)pentaerythritol-di-phosphite; tetrakis(2,4-di-tert-butylphenyl)4,4′-biphenylene diphosphonite; or a combination thereof.

The sulfur-based antioxidant may be dilauryl-3,3′-thiodipropionic acid ester; dimyristyl-3,3′-thiodipropionic acid ester; distearyl-3,3′-thiodipropionic acid ester; lauryl stearyl-3,3′-thiodipropionic acid ester; pentaerythrityl tetrakis(3-lauryl thiopropionic acid ester); or a combination thereof.

The phenol-based antioxidant may be included in an amount of 0.01 to 1 part by weight based on 100 parts by weight of the polyester resin.

The phosphorus-based antioxidant may be included in an amount of 0.01 to 1 part by weight based on 100 parts by weight of the polyester resin.

The sulfur-based antioxidant may be included in an amount of 0.01 to 1 part by weight based on 100 parts by weight of the polyester resin.

The sulfur-based antioxidant may be used in an amount of 30 parts by weight or more based on 100 parts by weight of the phenol-based antioxidant.

The phosphorus-based antioxidant and the sulfur-based antioxidant may be used in a weight ratio of 1:0.1 to 4, a weight ratio of 1:0.4 to 2.2, or a weight ratio of 1:0.8 to 1.2.

The phosphorus-based antioxidant and the phenol-based antioxidant may be used in a weight ratio of 1:0.1 to 4 or a weight ratio of 1:0.3 to 1.2.

The polyester film may include the antioxidant in an amount of 800 ppm or more, 1,000 ppm or more, 1,200 ppm or more, 1,800 ppm or more, 2,000 ppm or more, or 2,200 ppm or more based on 100 parts by weight of the polyester resin.

The antioxidant may be included in an amount of 5,000 ppm or less or 4,000 ppm or less. The use of the antioxidant in the above-described content can contribute to an improvement in the long-term durability of the film and can provide a film having excellent mechanical properties for a long period of time.

The inventors confirmed that the amount of the antioxidant applied to improve long-term durability may be associated with the monomer content of the resin. For example, the inventors confirmed that, when the polyester resin is a polycyclohexylenedimethylene terephthalate resin, and the amount of an isophthalic acid residue included in the resin is changed, there is a difference in the amount of a required antioxidant.

When C_{IPA} represents the content (mol %) of the isophthalic acid residue based on the total content of the dicarboxylic acid-based repeating unit in the polycyclohexylenedimethylene terephthalate resin, and C_{OA} represents the content of the antioxidant contained in the polyester film in ppm units (by weight), a long-term reliability index (units: ppm/mol %) is calculated by C_{OA}/(C_{IPA}+5).

The polyester film may have a long-term reliability index of 120 to 400 ppm/mol %, 130 to 350 ppm/mol %, or 180 to 300 ppm/mol %. When the long-term reliability index is very high, there are concerns about the waste of the antioxidant or the change in physical properties due to excessive use, and when the long-term reliability index is very low, long-term reliability may be degraded.

The polyester film may further include a pinning agent (electrostatic pinning agent).

As a pinning agent, an alkali metal salt or an alkaline earth metal salt may be used, and the pinning agent contributes to sheet formation of an extruded resin in a film manufacturing process. For example, as the pinning agent, a magnesium-based compound or a potassium-based compound may be used, and specifically, magnesium acetate, potassium acetate, or a mixture thereof may be used.

The pinning agent may be used so that the amount of the metal or metal ion included in the pinning agent is 300 to 1000 ppm based on 100 parts by weight of the polyester resin.

As a pinning agent, mixture of magnesium acetate and potassium acetate may be used. The mixture may include magnesium and potassium in a content ratio (molar ratio) of 1:1 to 10 or 1:5 to 10. Within the above-described range, the interaction of the pinning agent with other additives is substantially suppressed while the function of the pinning agent is sufficiently performed, and thus a film having improved durability can be provided.

The polyester film may have a thickness of 100 μm or less, 90 μm or less, 80 μm or less, 70 μm or less, 60 μm or less, or 50 μm or less. The polyester film may have a thickness of 1 μm or more, 2 μm or more, 5 μm or more, or 10 μm or more.

The polyester film has excellent long-term durability.

The polyester film may have a tensile strength of 7 kgf/mm² or more, 9 kgf/mm² or more, or 10 kgf/mm² or more as measured after being left at 250° C. for 30 minutes. The tensile strength may be 13 kgf/mm² or less or 11 kgf/mm² or less. The tensile strength is a value measured under harsh condition for evaluating long-term durability. Although the value may be slightly lower than that before the evaluation under harsh condition, it is a result showing excellent long-term durability and is superior to a general polyethylene terephthalate resin.

The polyester film may have a tensile strength of 10 kgf/mm² or more, 13 kgf/mm² or more, or 15 kgf/mm² or more as measured before the harsh test (being left at 250° C. for 30 minutes). The tensile strength may be 18 kgf/mm² or less.

The polyester film may have an elongation rate of 100% or more or 110% or more as measured after being left at 250° C. for 30 minutes. The elongation rate may be 140% or less or 135% or less. This means that excellent long-term durability is exhibited in terms of an elongation rate among mechanical properties.

A general polyethylene terephthalate resin exhibits a substantially degraded elongation rate after being left under the harsh condition of 250° C. for 30 minutes, and a polyethylene naphthalate (PEN) film having relatively excellent mechanical strength also exhibits a substantially decreased elongation rate after being left under the harsh condition of 250° C. for 30 minutes, whereas the polyester film according to an embodiment shows an excellent result compared to the two films.

The polyester film may have an insignificant modulus (elastic modulus) variation, which is within 10%, before and after being left at 250° C. for 30 minutes. This means that the modulus value is maintained well even after evaluation of long-term durability.

The polyester film may have a modulus of 200 kgf/mm² or more, 210 kgf/mm² or more, or 220 kgf/mm² or more. The modulus may be 400 kgf/mm² or less, 300 kgf/mm² or less, or 280 kgf/mm² or less.

The polyester film may have a haze value of 15% or less, 12% or less, 10% or less, or 8% or less as measured after being left at 250° C. for 30 minutes. The haze value may be 1% or more.

The polyester film may have a haze value of 8% or less or 6% or less. The haze value may be 1% or more.

As compared to a PEN film known to have excellent heat resistance or a general polyethylene terephthalate resin, the polyester film according to an embodiment may have excellent haze characteristics.

The polyester film may have a yellow index of 3 or less or 2 or less as measured after being left at 250° C. for 30 minutes. The yellow index may be 0.1 or more.

The polyester film may have a yellow index of 0.1 or more or 0.3 or more and 1 or less.

The polyester film may have an intrinsic viscosity (I.V.) variation before and after being left at 250° C. for 30 minutes within 10%. The intrinsic viscosity of a polymer material may be one of the criteria for evaluating weatherability, and an intrinsic viscosity value usually decreases as a film ages. The polyester film according to an embodiment may have an insignificant I.V. variation within 10% even after the harsh test of being allowed to stand at 250° C. for 30 minutes. This means that excellent long-term durability is exhibited.

Measurement of the above-described properties such as mechanical strength, yellow index, haze value, intrinsic viscosity, and the like is made by a method applied in measurement in examples to be described below.

The polyester film may have a machine direction (MD) shrinkage rate of 1.2% to 2.05% or 1.5% to 2% after being left under the hot-air condition of 150° C. for 30 minutes. The polyester film may have a transverse direction (TD) shrinkage rate of 0.1% to 0.7% or 0.2% to 0.6% after being left under the hot-air condition of 150° C. for 30 minutes. As the polyester film has the above-described MD and TD shrinkage rates, the deterioration or detachment of the film at high temperature can be minimized.

The shrinkage rate may be calculated as follows.

Shrinkage rate (%)={(Original length−Length after shrinkage)/(Original length)}×100% [Equation 1]

The polyester film including a repeating unit having a cyclohexane skeleton in the diol-based repeating unit and a repeating unit having an isophthalic acid residue in the dicarboxylic acid-based repeating unit shows superior for application in an environment in which exposure to heat and moisture is repeated due to having excellent moisture resistance and heat resistance. The application of the polyester film according to an embodiment can be increased by improving mechanical strength through a process such as stretching and the like, improving mechanical properties using various technical means such as adjustment of a monomer ratio, application of an additive such as an antioxidant, and the like, and maintaining the improved mechanical strength and mechanical properties for a long period of time.

Since the polyester film having the above characteristics has a suitably controlled degree of crystallization according to heat and/or orientation for application as a film, a polyester film having excellent mechanical properties can be manufactured, and workability can also be improved in a film manufacturing process. Particularly, as the orientation of a polymer chain is changed in a stretching process, and heat application is repeated in the process, the crystallinity of a polymer resin may be changed. Particularly, in the case of a polymer resin whose crystallinity is rapidly changed, processing into a film and control are difficult. However, an embodiment can help control the polymer in the processing process.

Method of Manufacturing Polyester Film

A method of manufacturing a polyester film according to yet another embodiment includes: extruding a polyester resin composition to prepare a sheet (extrusion step); stretching the sheet to prepare a film before thermal treatment (stretching step); and thermosetting the film before thermal treatment to prepare a polyester film (thermosetting step).

In at least one of the extrusion step, the stretching step, and the thermosetting step, a process condition for adjusting the degree of crystallization of the polyester film is set based on the degree of crystallization of the polyester film confirmed using a Fourier transform infrared spectrometer.

The polyester film includes a polyester resin.

The polyester resin includes a repeating unit having a cyclohexane skeleton and a repeating unit having a benzene ring.

The polyester film has a base peak which is a peak related to bending vibration of the benzene ring and a first peak which is a peak related to C—O stretching vibration in a spectrum measured using a Fourier transform infrared spectrometer (FT-IR).

The base peak is a peak at a wavenumber of 788 cm⁻¹ to 798 cm⁻¹.

The first peak is a peak at a wavenumber of 1129 cm⁻¹ to 1139 cm⁻¹.

The intensity of the base peak and the intensity of the first peak differ by −0.05 to +0.05.

The manufacturing method according to an embodiment may adjust the FT-IR spectrum characteristics of a polyester film by controlling the process conditions for the extrusion step, the stretching step, and the thermosetting step. Accordingly, the thermal crystallization characteristics and orientation characteristics of a polyester film may be simultaneously controlled to provide a film having stable flexibility and mechanical properties while having excellent heat resistance.

The polyester resin composition may include a polyester resin. Since the description of the polyester resin is the same as that described above, a detailed description thereof is omitted.

The polyester resin may be prepared by condensation polymerization of a diol-based compound and a dicarboxylic acid-based compound, but the present invention is not limited thereto.

The polyester resin composition may further include an additive including inorganic particles, an antioxidant, a pinning agent, and the like. Since the description of the additive is the same as that described above, a detailed description thereof is omitted.

In the extrusion step, a polyester resin composition may be melt-extruded using an extruder. The extruder may be a single-screw extruder. The extruder may be a twin-screw extruder.

In the extrusion step, a melt extrusion temperature may be 270° C. to 330° C. The melt extrusion temperature may be 275° C. or more. The melt extrusion temperature may be 280° C. or more. The melt extrusion temperature may be 320° C. or less. The melt extrusion temperature may be 310° C. or less. In this case, a certain level or more of orientation can be imparted to the extruded resin composition while deterioration of the resin due to high temperature is suppressed.

The extruded resin composition may be cooled and shaped to prepare a sheet. Specifically, a resin composition may be melt-extruded using a typical T-die and brought into close contact with a cooling roll to prepare a sheet.

A cooling temperature of the cooling roll may be 10° C. to 30° C. The cooling temperature may be 12° C. or more. The cooling temperature may be 15° C. or more. The cooling temperature may be 27° C. or less. The cooling temperature may be 25° C. or less. In this case, excessive crystallization of a resin in the unstretched sheet can be effectively suppressed.

The stretching step may include a longitudinal stretching process of longitudinally stretching the sheet to form a longitudinally stretched sheet and a lateral stretching process of laterally stretching the longitudinally stretched sheet to prepare a film before thermal treatment.

In the longitudinal stretching process, the unstretched sheet may be preheated to 70° C. to 85° C., and the preheated unstretched sheet may be longitudinally stretched while moving at 10 m/min to 50 m/min.

A stretching temperature in the longitudinal stretching process may be adjusted. The stretching temperature is a temperature of a stretching roll.

The stretching temperature in the longitudinal stretching process may be 75° C. to 110° C. The stretching temperature may be 80° C. or more. The stretching temperature may be 100° C. or less. In this case, an excessive increase in the crystallization rate of the polyester resin due to heat generated in the stretching process can be suppressed, and the longitudinal orientation of the polyester resin can be improved.

In the lateral stretching process, the longitudinally stretched sheet may be preheated to 80° C. to 120° C., and the preheated sheet may be laterally stretched at 85° C. to 140° C. to form a film before thermal treatment.

In the stretching step of an embodiment, the stretching ratios of the longitudinal stretching process and the lateral stretching process for adjusting the crystallization characteristics of the polyester film may be set based on the spectrum of the polyester film confirmed using a Fourier transform infrared spectrometer. In other words, the stretching ratio of the longitudinal stretching process and the stretching ratio of the lateral stretching process may be set so that the manufactured polyester film has the above-described FT-IR spectrum distribution characteristics.

Since the description of the FT-IR spectrum distribution characteristics of the polyester film is the same as that described above, a detailed description thereof is omitted.

Accordingly, the orientational crystallinity of the manufactured polyester film may be more sophisticatedly controlled.

A stretching ratio applied in the longitudinal stretching may be in a range of 2 to 4. The stretching ratio may be 2.5 or more. The stretching ratio may be 2.8 or more. The stretching ratio may be 3.5 or less. The stretching ratio may be 3.3 or less.

A stretching ratio applied in the lateral stretching may be in a range of 3 to 5. The stretching ratio may be 3.5 or more. The stretching ratio may be 4.5 or less.

In this case, the orientational crystallinity of the manufactured film may be controlled to an appropriate level to impart stable flexibility and mechanical strength to the film.

In the thermosetting step of an embodiment, a thermosetting temperature for adjusting the crystallization characteristics of the polyester film may be set based on the spectrum of the polyester film confirmed using a Fourier transform infrared spectrometer. In other word, the thermosetting temperature may be set so that the manufactured polyester film has the above-described FT-IR spectrum distribution characteristics. Since the description of the FT-IR spectrum distribution characteristics of the polyester film is the same as that described above, a detailed description thereof is omitted.

Accordingly, excellent dimensional stability can be imparted to the film while thermal crystallization of the manufactured film at an excessively high temperature is suppressed.

In the thermosetting step, a thermosetting temperature may be 200° C. to 250° C. The thermosetting temperature may be 210° C. or more. The thermosetting temperature may be 220° C. or more. The thermosetting temperature may be 245° C. or less. The thermosetting temperature may be 240° C. or less. In this case, dimensional stability of the film can be effectively increased while excessive crystallization of the polyester film due to heat is suppressed.

A polyester film manufactured by the manufacturing method according to an embodiment may have the same characteristics as those of the above-described polyester film. Since the description of the polyester film manufactured by the manufacturing method according to an embodiment is the same as that described above, a detailed description thereof is omitted.

When the polyester film has FT-IR spectrum characteristics in a predetermined range in an embodiment, crystallization characteristics of the film may be more sophisticatedly controlled. Such a film can obtain both flexibility and high mechanical strength while having high heat resistance.

Hereinafter, the present invention will be described in further detail with reference to specific examples. The following examples are provided only to promote understanding of the present invention, and the scope of the present invention is not limited to these examples.

1. Manufacture of Polyester Resin and Sequentially Biaxially Stretched Film

100 mol % of cyclohexanedimethanol (CHDM) as a diol-based compound and a monomer mixture including 96 mol % of terephthalic acid (TPA) and 4 mol % of isophthalic acid (IPA) as a dicarboxylic acid-based compound were input into a stirrer, a titanium catalyst was added in an amount of 1 ppm based on 100 parts by weight of the resulting mixture, and a transesterification reaction was performed at 275° C.

The product of the transesterification reaction was transferred to a separate reactor equipped with vacuum equipment and then polymerized at 285° C. for 160 minutes to obtain a polycyclohexylenedimethylene terephthalate (PCT) resin.

The PCT resin was processed in the form of a chip, and the chip was mixed with an pinning agent including a Mg-based pinning agent and a K-based pinning agent in a ratio of 9:1 based on a metal weight to prepare a pinning agent+polymer chip, mixed with a filler including bulk silica having a size of 3.8 μm to prepare a filler+polymer chip, and mixed with an antioxidant including Irganox 1010 (manufactured by BASF), Irgafos 168 (manufactured by BASF), and AO-412S (manufactured by ADEKA) in a weight ratio of 4:2:4 to prepare an antioxidant+polymer chip.

The chips were mixed so that the pinning agent was 300 ppm (based on a weight), the filler was 375 ppm (based on a weight), and the antioxidant was 2000 ppm (based on a weight) based on the entire film, then dried at about 140° C., input into an extruder, and shaped in the form of a sheet by melting at about 280 to 300° C. Afterward, the sheet was stretched in a machine direction (MD) at 80 to 95° C. and in a transverse direction (TD) at 90 to 130° C. and thermoset at 200 to 240° C. to prepare a sequentially biaxially stretched film. The stretching ratio and thermosetting temperature of each film sample are shown in Table 1 below. The degree of crystallization of the unstretched sheet was also evaluated below.

2. Preparation of Sample

(1) Sample for Measuring Degree of Thermal Crystallization

Each film sample was cut so that each corner was at right angles. The length of one side was about 5 to 6 cm.

(2) Sample for Measuring Degree of Orientational Crystallization

A sample was prepared in the same manner as in (1), except that the sample was cut to a length of 5 to 6 cm so that the right angles of the corners were exactly parallel to the MD and TD.

3. Measurement of Spectrum

(1) Measurement of the Degree of Surface Orientation

The sample was cut to a length and width of 5 to 6 cm so that the corners of the sample were at right angles to prepare a sample.

As a FT-IR instrument, a UMA 600 product manufactured by Varian was used. In the instrument, the Resolutions Pro software was set to collect-rapid scan, and the background was measured after beam calibration. Diamond and Ge single bounce ATR was applied, and conditions of an MCT detector, resolution of 4 cm⁻¹, and #32 scans were applied. Measurement was made in a wavenumber range of about 800 to about 1200 cm⁻¹. In the case of the bench mode, since the IR beam was not perpendicularly incident on the sample, different spectrum intensity values were measured at the same wavenumber according to a sample direction. Therefore, after measurement in two directions perpendicular to each other, the average thereof was used.

(2) Measurement of Degree of Directional Orientation

The sample was cut to a length and width of 5 to 6 cm so that two sides extending from the corner of the sample were parallel to the machine and transverse directions to prepare a sample.

The same instrument and setup as described above were applied, a polarizing plate was mounted between a light source and the sample and set to 90°, and the position thereof was adjusted so that an interferogram signal was maximized. When the polarizing plate was set to 90°, the direction of the wavelength of the light beam was parallel to the surface of the sample, and the light beam passing through the sample enabled confirmation of the MD crystallinity of the film perpendicular to the traveling direction. Also, when the direction of the wavelength of the light beam was parallel to the thickness of the sample by adjusting the polarizing plate, the light beam passing through the sample enabled confirmation of the TD crystallinity of the film perpendicular to the traveling direction. A sensitivity of 8 was applied.

Measurement results are shown in Table 1 below.

(3) Measurement of Degree of Crystallization by X-Ray Diffraction (XRD) and Differential Scanning Calorimetry (DSC)

In the case of XRD, the sample was irradiated with X-rays using an Ultima IV instrument manufactured by Rigaku, and then the diffraction angle of the diffracted x-rays was measured to obtain data about a degree of crystallization. As measurement conditions, a tube voltage of 20 to 60 kV, a tube current of 2 to 60 mA, a 26 measuring range of −3° to 162°, and a minimum step size of 0.0001° were applied.

In the case of DSC, a Q2000/Q1000 instrument manufactured by TA Instruments was used. As measurement conditions, a temperature range of −180 to 725° C., a temperature accuracy of ±0.1° C., a scanning rate of <+200° C./min/<-100° C./min, and a max sensitivity of 0.2 μW were applied, and modulated DSC and an autosampler were used for measurement.

After measurement, the evaluated degree of crystallization was shown in Table 1 below.

(4) Difference Between Search Intensity and Base Intensity

Among peaks in the spectrum measured using the FT-IR instrument, a case in which a difference between the intensity of a peak at a base wavenumber of 793 cm⁻¹ and the intensity of each peak at first to fourth wavenumbers was in a range of −0.05 to +0.05 was represented by ∘, and a case in which the difference was in a range of −0.02 to +0.02 was represented by ⊚. A case in which the difference was outside the above-described ranges was represented by X. Evaluation results are shown in Table 2.

TABLE 1

| | | | FT-IR spectrum intensity | | | | |
|---|---|---|---|---|---|---|---|
| | | | | Third | | | |
| | | | | wavenumber | | | |
| | Stretching | Thermosetting | First | 855 cm⁻¹ | Second | DSC | XRD |
| | ratio | temperature | wavenumber | [MD/TD | wavenumber | Degree of | |
| | MD × TD | ° C. | 1134 cm⁻¹ | directionality] | 815 cm⁻¹ | crystallization | |
| Sample 1 | — | — | −0.81 | −0.1 | −0.05 | — | — |
| Sample 2 | 3.0 × 3.8 | 200 | 0.8 | 1.06 | 0.31 | 42.7 | 63.16 |
| Sample 3 | 3.0 × 3.8 | 230 | 0.98 | 1.11 | 0.44 | 42.62 | 63.65 |
| Sample 4 | 3.0 × 3.5 | 230 | 1.33 | 1.44/1.19 | 0.48 | 43.42 | 65.13 |
| Sample 5 | 3.2 × 3.5 | 230 | 1.32 | 1.35/1.23 | 0.49 | 42.85 | 64.84 |
| Sample 6 | 3.0 × 3.9 | 230 | 1.33 | 1.53/1.13 | 0.47 | 42.56 | 65.13 |
| Sample 7 | 3.2 × 3.9 | 230 | 1.34 | 1.47/1.17 | 0.48 | 42.5 | 65.43 |

TABLE 2

| | Difference | Difference | Difference | Difference |
|---|---|---|---|---|
| | between | between | between | between |
| | first | second | third | fourth |
| | intensity | intensity | intensity | intensity |
| | and base | and base | and base | and base |
| | intensity | intensity | intensity | intensity |
| Sample 1 | X | X | X | X |
| Sample 2 | ⊚ | ⊚ | ⊚ | ⊚ |
| Sample 3 | ⊚ | ⊚ | ⊚ | ⊚ |
| Sample 4 | ⊚ | ⊚ | ⊚ | ⊚ |
| Sample 5 | ⊚ | ⊚ | ⊚ | ⊚ |
| Sample 6 | ⊚ | ⊚ | ⊚ | ⊚ |
| Sample 7 | ⊚ | ⊚ | ⊚ | ⊚ |

Sample 1 is a result obtained by measuring the unstretched sheet, and Samples 2 and 3 are results obtained by measuring the samples under the same conditions except that thermosetting temperatures of 200° C. and 230° C. were applied.

Referring to the results of Samples 2 and 3, the DSC or XRD results did not show a significant change according to a thermosetting temperature, whereas the FT-IR results showed a significant change and a tendency in the intensity at a first wavenumber of 1134 cm⁻¹, the intensity at a second wavenumber of 815 cm⁻¹, and the intensity at a third wavenumber of 855 cm⁻¹ according to a thermosetting temperature. Although only two results were presented as a representative example, reproducibility was confirmed by repeated evaluation at various temperatures (the same as below).

Samples 3 to 7 are measurement results obtained by varying a stretching ratio while maintaining a thermosetting temperature. Samples 3 to 7 were measured using the sample for measuring a degree of orientational crystallization. Referring to the results of Samples 3 to 7, the intensity at a first wavenumber of 1134 cm⁻¹ and the intensity at a second wavenumber of 815 cm⁻¹ were almost constant, which results from the constant application of a thermosetting temperature, and reliability which is a characteristic for confirming the degree of crystallization by heat was high. On the other hand, the DSC or XRD results were judged to be inappropriate for use as a characteristic for confirming the degree of crystallization because the values fluctuated and the tendency was not clear.

From the result of measuring the intensity at a third wavenumber of 855 cm⁻¹, it was confirmed that FT-IR spectrum intensity tended to change according to a degree of MD and TD stretching. For example, when a MD stretching ratio was fixed to 3, a tendency in which MD intensity increased and TD intensity decreased as a TD stretching ratio was increased from 3.5 to 3.9 was shown. Also, when a MD stretching ratio was fixed to 3.2, the same tendency was shown. On the other hand, as a MD stretching ratio was increased from 3 to 3.2 while a TD stretching ratio was fixed to 3.5, TD intensity increased, and MD intensity decreased. This shows that the tendency of orientational crystallinity could be confirmed from the FT-IR spectrum intensity at a third wavenumber.

It is thought that the degree of crystallization of a polymer can be effectively confirmed or predicted using these characteristics.

On the other hand, in the case of the degree of crystallization measured by DSC, it was difficult to confirm a tendency according to thermal crystallization or orientational crystallization, and in the case of the degree of crystallization measured by XRD, the confirmation of the tendency was also difficult.

A polyester film manufactured in this way could be well prepared into a biaxially stretched film due to having a controlled degree of crystallization, and spectrum characteristics measured using the manufactured films were also confirmed.

While exemplary embodiments of the present invention have been described above in detail, the scope of the present invention is not limited thereto, and encompasses several modifications and improvements by those skilled in the art using basic concepts of embodiments of the present invention defined by the appended claims.

What is claimed is:

1. A polyester film comprising a polyester resin,
wherein the polyester resin comprises a repeating unit having a cyclohexane skeleton and a repeating unit having a benzene ring,
wherein the polyester film has a base peak which is a peak related to bending vibration of the benzene ring and a first peak which is a peak related to C—O stretching vibration in a spectrum measured using a Fourier transform infrared spectrometer (FT-IR),
wherein the base peak is a peak at a wavenumber of 788 cm⁻¹ to 798 cm⁻¹,
wherein the first peak is a peak at a wavenumber of 1129 cm⁻¹ to 1139 cm⁻¹, and
an intensity of the base peak and an intensity of the first peak differ by −0.05 to +0.05.

2. The polyester film of claim 1, which has a second peak which is a peak distinguished from the first peak and related to C—O stretching vibration in the spectrum,
wherein the second peak is a peak at a wavenumber of 810 cm⁻¹ to 820 cm⁻¹, and
the intensity of the base peak and an intensity of the second peak differ by −0.05 to +0.05.

3. The polyester film of claim 1, which has a third peak which is a peak related to C═O—O stretching vibration in the spectrum,
wherein the third peak is a peak at a wavenumber of 850 cm⁻¹ to 860 cm⁻¹, and
the intensity of the base peak and an intensity of the third peak differ by −0.05 to +0.05.

4. The polyester film of claim 1, which has a fourth peak which is a peak related to C═O—O stretching vibration in the spectrum,
wherein the fourth peak is a peak at a wavenumber of 966 cm⁻¹ to 976 cm⁻¹, and
the intensity of the base peak and an intensity of the fourth peak differ by −0.05 to +0.05.

5. The polyester film of claim 1, which has a tensile strength of 7 kgf/mm² or more as measured after being left at 250° C. for 30 minutes.

6. The polyester film of claim 1, which has an elongation rate of 100% or more as measured after being left at 250° C. for 30 minutes.

7. The polyester film of claim 1, which has a haze value of 15% or less as measured after being left at 250° C. for 30 minutes.

8. The polyester film of claim 1, which has a yellow index of 3 or less as measured after being left at 250° C. for 30 minutes.

9. The polyester film of claim 1, wherein the repeating unit having a benzene ring comprises a dicarboxylic acid-based repeating unit, and
wherein the dicarboxylic acid-based repeating unit comprises a terephthalic acid-based repeating unit and an isophthalic acid-based repeating unit.

10. The polyester film of claim 1, wherein the polyester resin has a weight-average molecular weight of 30,000 g/mol to 50,000 g/mol.

11. A method of manufacturing a polyester film, comprising:
extruding a polyester resin composition to prepare a sheet (extrusion step);
stretching the sheet to prepare a film before thermal treatment (stretching step); and
thermosetting the film before thermal treatment to prepare a polyester film (thermosetting step),
wherein the polyester film comprises a polyester resin,
wherein the polyester resin comprises a repeating unit having a cyclohexane skeleton and a repeating unit having a benzene ring,
wherein the polyester film has a base peak which is a peak related to bending vibration of the benzene ring and a first peak which is a peak related to C—O stretching vibration in a spectrum measured using a Fourier transform infrared spectrometer (FT-IR),
wherein the base peak is a peak at a wavenumber of 788 cm⁻¹ to 798 cm⁻¹,
wherein the first peak is a peak at a wavenumber of 1129 cm⁻¹ to 1139 cm⁻¹,
wherein the polyester film has a difference in an intensities of the base peak and the first peak of −0.05 to +0.05, and
in at least one of the extrusion step, the stretching step, and the thermosetting step, a process condition for adjusting the crystallization characteristics of the polyester film is set based on the spectrum of the polyester film measured using a Fourier transform infrared spectrometer.

12. The method of claim 11, wherein the stretching step comprises a longitudinal stretching process of longitudinally stretching the sheet to prepare a longitudinally stretched sheet and a lateral stretching process of laterally stretching the longitudinally stretched sheet to prepare a film before thermal treatment,
in the stretching step, stretching ratios of the longitudinal stretching process and the lateral stretching process for adjusting the crystallization characteristic of the polyester film are set based on the spectrum of the polyester film measured using a Fourier transform infrared spectrometer, and
the stretching ratio of the longitudinal stretching process is in a range of 2 to 4, and the stretching ratio of the lateral stretching process is in a range of 3 to 5.

13. The method of claim 11, wherein, in the thermosetting step, a thermosetting temperature for adjusting the crystallization characteristics of the polyester film is set based on the spectrum of the polyester film measured using a Fourier transform infrared spectrometer, and
the thermosetting temperature is 200° C. to 250° C.
